(19) United States
(12) Patent Application Publication
(10) Pub. No.: US 2019/0093098 A1
(43) Pub. Date: Mar. 28, 2019

(54) METHODS AND SUBSTANCES FOR DIRECTED RNA EDITING

(71) Applicant: Eberhard Karls Universität Tübingen, Tübingen (DE)

(72) Inventors: Thorsten Stafforst, Tübingen (DE); Jacqueline Wettengel, Tübingen (DE); Paul Vogel, Tübingen (DE)

(21) Appl. No.: 15/763,351

(22) PCT Filed: Aug. 9, 2016
(86) PCT No.: PCT/DE2016/000309

§ 371 Date: Mar. 26, 2018

(30) Foreign Application Priority Data
Sep. 26, 2015 (DE) 102015012522.2

(51) Int. Cl. C12N 15/10; C12N 15/113

(52) U.S. Cl. CPC C12N 15/102; C12N 15/113; C12Y 305/04004; C12N 2310/20; C12N 2320/34; C12N 2310/531

(57) ABSTRACT

The invention relates to methods and substances for the targeted alteration of genetic information on an RNA level. The substances are artificially produced guide RNAs, which are capable of recruiting endogenous editing enzymes, such as hADAR enzymes, in particular hADAR2 and hADAR1, in order to introduce targeted point mutations in selected mRNAs. The guide RNA consists of multiple segments and is constructed in such a way that individual nucleotides from different segments pair to form a double helix, and the nucleotides of a determined segment form a hairpin structure within the guide RNA. The invention also relates to the method for directed RNA editing, wherein the guide RNA is transfected into the cells in which the RNA editing is to be carried out. The substances and the method can be used for repairing individual, e.g. disease-relevant point mutations, such as those leading to premature stop signals. An advantage of the invention is that endogenous editing enzymes are also used in order to introduce targeted point mutations into the RNA. Only the short guide RNA, used for recruiting endogenous editing enzymes, must be artificially produced for each specific problem and ectopically expressed.

### SEQUENCE LISTING

This application contains a sequence listing in accordance with 37 C.F.R. 1.821-1.825. The sequence listing accompanying this application is hereby incorporated by reference in its entirety.

### FIELD OF THE INVENTION

The present invention relates to a method and substances for targeted alteration of genetic information on the RNA level.

The nucleoside adenosine can be enzymatically desaminated. Inosine is created, and during a translation is read as guanosine. This nucleoside alteration is called A-to-I editing and may be used to insert individual point mutations into RNA to alter the resulting proteins in a targeted manner.

### BACKGROUND OF THE INVENTION

It is known of the naturally occurring hADARs (human adenosine deaminase acting on RNA) enzymes that they can desaminate adenosine and are highly promiscuous therein (5). Thus the enzyme hADAR2 can detect and edit thousands of different substrates, wherein nearly every double strand RNA having a length of more than 30 base pairs can act as a substrate. Nevertheless, only a very few substrates are edited highly selectively and efficiently (7). The reason for this seems to be the recognition of special sequences and secondary structures by the dsRBD (dsRNA binding domains). ADAR2 has two dsRBD (1 & 2) in the N-terminus. It was possible to demonstrate on model substrates, such as the GluR2 transcript, that the dsRBD1 & 2 each bind at different regions in the GluR2 transcript in defined modes (8, FIG. 1). The deaminase domain is placed at the C-terminal end of hADAR2.

Known from the prior art is a method for directing its enzyme activity, in a highly specific and RNA-dependent manner, to new substrates (1, 2, 4, 6, 9). The dsRBD-protein domains are replaced by a so-called SNAP tag to which a specially constructed guide RNA is covalently coupled. The aforesaid SNAP tag construct is activated by this specially constructed added guide RNA.

In another known method, an in-peptide is added to the natural hADAR2 and a BoxB motif is added to the guide RNA to enable the binding (3).

One major drawback of the methods known from the prior art is that they do not use the naturally occurring ADAR2 enzyme, but the enzyme variant modified with the SNAP tag or in-peptide. Another drawback is that the guide RNA includes chemically altered nucleotides, such as, e.g., benzylguanine (BG), that are not genetically codable. In these methods, therefore, the guide RNA must first be produced in vitro and may only be transfected to the cell afterwards.

### SUMMARY OF THE INVENTION

The object of the present invention is to provide a method and substances with which it is possible to add point mutations in gene products (RNA) efficiently and reversibly without altering the coding gene.

This object is attained by provided specially constructed, genetically codable guide RNAs that are capable of recruiting endogenous editing enzymes in order to introduce in a targeted manner point mutations in selected RNAs.

Specifically, the inventive guide RNA includes at least the following nucleotides that are coupled to one another, listed from the 5′ end (FIG. 2):
Segment A: Nucleotide sequence having a length of three to five bases, wherein a guanosine is always in position 1 and a uridine is always in position 2;
Segment B: Nucleotide sequence having a length of three to five bases, wherein an adenosine is always in position 1;
Segment C: Nucleotide sequence having a length of eight to ten bases, wherein a guanosine is always in position 1;
Segment D: Nucleotide sequence UAUGCUAAAUG or UAUGCUCAAUG;
Segment E: Nucleotide sequence having a length of eight to ten bases, wherein a guanosine is always in the last position;
Segment F: Nucleotide sequence having a length of three to five bases, wherein a cytosine is always in the last position;
Segment G: Nucleotide sequence having a length of three to five bases, wherein an adenosine is always in the next-to-the-last position and a cytosine is in the last position;
Segment H: Nucleotide sequence having a length of five to nine bases, wherein a cytosine or a uridine is always in the last position;
Segment I: Nucleotide sequence having a length of eight to twenty bases.

Therein, individual nucleotides of the segments A and G, B and F, or C and E pair to form a double helix and the nucleotides of segment D form a hairpin structure (FIG. 2).

Segment A has in particular the length of four nucleotides and has the GUGG nucleotide sequence.

Segment B has in particular the length of four nucleotides and has the AAUA nucleotide sequence.

Segment C has in particular the length of nine nucleotides and has the GUAUAACAA sequence.

Segment E has in particular the length of nine nucleotides and has the UUGUUAUAG nucleotide sequence.

Segment F has in particular the length of four nucleotides and has the UAUC nucleotide sequence.

Segment G has in particular the length of four nucleotides and has the CCAC nucleotide sequence.

Segments H and I are designed such that they pair with the target mRNA and place the target base to be edited in an A:C mismatch pair.

The preferred guide RNAs are shown in the SEQ ID NO 1 through SEQ ID NO 7 sequences, which are given in the sequence listing.

In one special embodiment of the invention, the guide RNA is appended on the 3′ end for stabilizing additionally a hairpin structure, e.g. a BoxB motif.

The structure of the particularly preferred embodiment of the inventive guide RNA is illustrated in FIG. 3.

The editing enzymes involved are primarily hADAR enzymes, in particular the hADAR2 and hADAR1 enzymes. Since the two enzymes are involved with different substrates and are expressed differently in body tissues, according to the invention specific tissues or cells may be targeted using the selection of the editing enzymes.

It is possible to repair individual point mutations, such as, for instance, those that lead to premature stop signals, by means of a nucleotide exchange attained in this manner.

The nucleotide exchange may be attained at one or at a plurality of positions of the target RNA, so that even multi-genetic diseases may be treated with the inventive method.

According to the invention, therapy-relevant guide RNAs are attained either using individual administrations to the patient, e.g., are administered to the affected organs or cells, or they may be continuously expressed using installation in the cells or organs of the patient. The therapeutic guide RNA may be transferred, e.g., virally, as stabilized mRNA, or genetically coded. The desired point mutations may be switched tissue-specific, inducible, or reversible. Likewise, the degree of editing may be set according to need and may be adjusted depending on specific circumstances and therapy goals, so that editing may be attained to a degree between 0 and 100%.

Since in mammals the ADAR deaminases are strongly expressed especially in neurons, the inventive method may be employed for treating neurological diseases that are caused, for example, by individual point mutations. The intrinsically present ADARs could be recruited for corrective RNA editing using a single administration or ectopic expression of small artificial guide RNAs.

It is a major advantage of the inventive method that endogenous editing enzymes, i.e., the cell's own editing enzymes, are used to introduce targeted point mutations to the RNA. Only the short guide RNA, which is used according to the invention for recruiting endogenous editing enzymes, must be artificially produced for each special problem and expressed ectopically. Because of this, the targeted alteration of proteins on the translational level is particularly simple, so that it is possible to correct point mutations causing disease. In contrast to the prior art, with the inventive method it is not necessary to express an additional, exogenous protein, which is highly advantageous, especially for medical applications. Efficiency is also higher and there are fewer adverse secondary effects because only one construct has to be transferred to the target tissue, rather than two or more.

The method is particularly simple and advantageous due to the codability of the constructs used, because there is no synthesis of chemically stabilized guide RNAs: the guide RNA is coded on a plasmid, transfected into the cell, and formed by the cell itself.

Additional advantages, features, and potential applications of the invention shall be described in the following using the exemplary embodiments described below, and referring to the figures.

### BRIEF DESCRIPTION OF THE DRAWINGS

In the figures, the broken line represents the constructed inventive guide RNA, while the solid line represents the target mRNA to be edited; the asterisk marks the nucleotide to be edited in the target mRNA.

FIG. 1: depicts the structure of a natural editing motif of the GluR2 transcript with the bound ADAR2, which has one deaminase domain and two dsRNA binding domains (dsRBD1 and dsRBD2).

FIG. 2: depicts the general structure of the inventive guide RNA. The letters A through I mark the individual segments of the guide RNA;

FIG. 3: depicts the structure of the preferred embodiment of the inventive guide RNA;

FIG. 4: depicts the structure of the constructed guide RNA for the targeted editing of W58X stop codon in the GFP gene as an exemplary embodiment according to the SEQ ID NO 1;

FIG. 5: depicts the structure of the constructed guide RNA for the targeted editing of W58X stop codon in the GFP gene as an exemplary embodiment according to the SEQ ID NO 3.

FIG. 6: depicts the structure of the constructed guide RNA for the targeted editing of the R407Q mutation in the “CAG” codon in the PINK1 gene as an exemplary embodiment;

FIG. 7: depicts the structure of the constructed guide RNA for the targeted editing of the W437X mutation in the “UAG” codon in the PINK1 gene as an exemplary embodiment;

FIG. 8: depicts the structure of the constructed guide RNA for the targeted editing of the W417X mutation in the “UAG” codon in the luciferase gene as an exemplary embodiment;

FIG. 9: depicts the structure of the constructed guide RNA for the targeted editing of a disease-relevant mutation R521H in the “CAC” codon in the FUS gene as an exemplary embodiment.

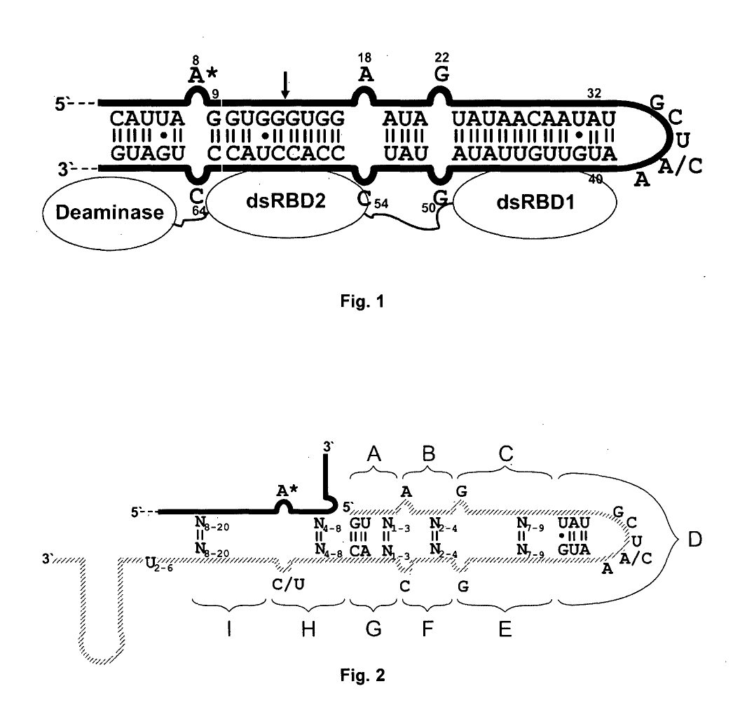

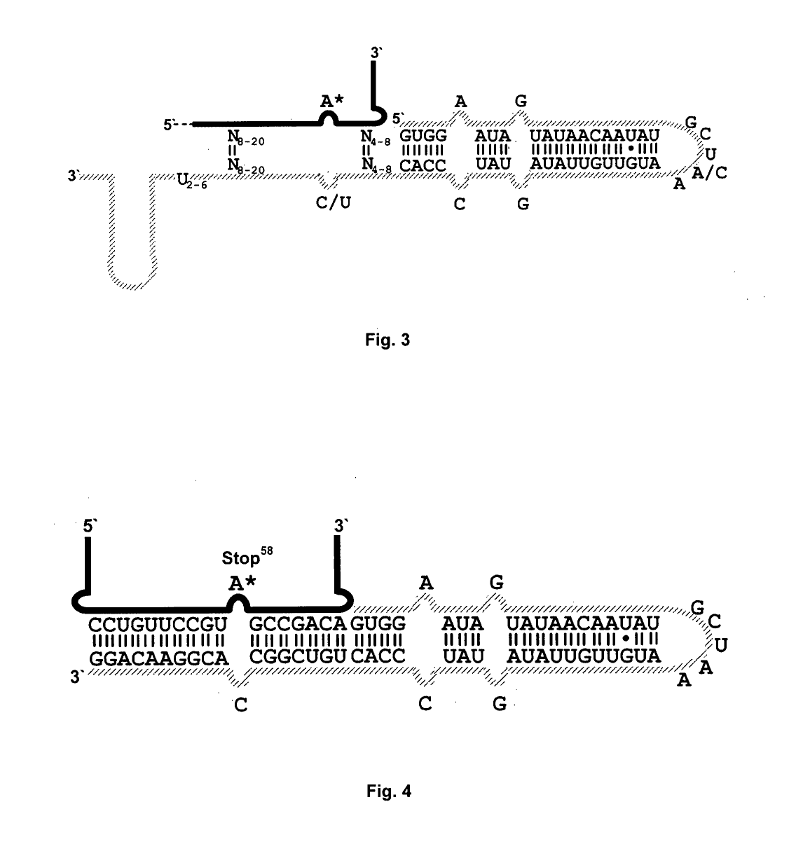

### DETAILED DESCRIPTION OF THE INVENTION

HEK 293T cells were used for transfecting the guide RNA according to the invention. For each experiment, 1.75*10≡cells were prepared in a 24-well plate format on the day prior to the transfection. Plasmids that are specific for human cell lines were used for the experiments. Lipofectamine™ 2000 (Invitrogen) was used as the transfection reagent.

### Targeted Editing of W58X Stop Codon in the GFP Gene

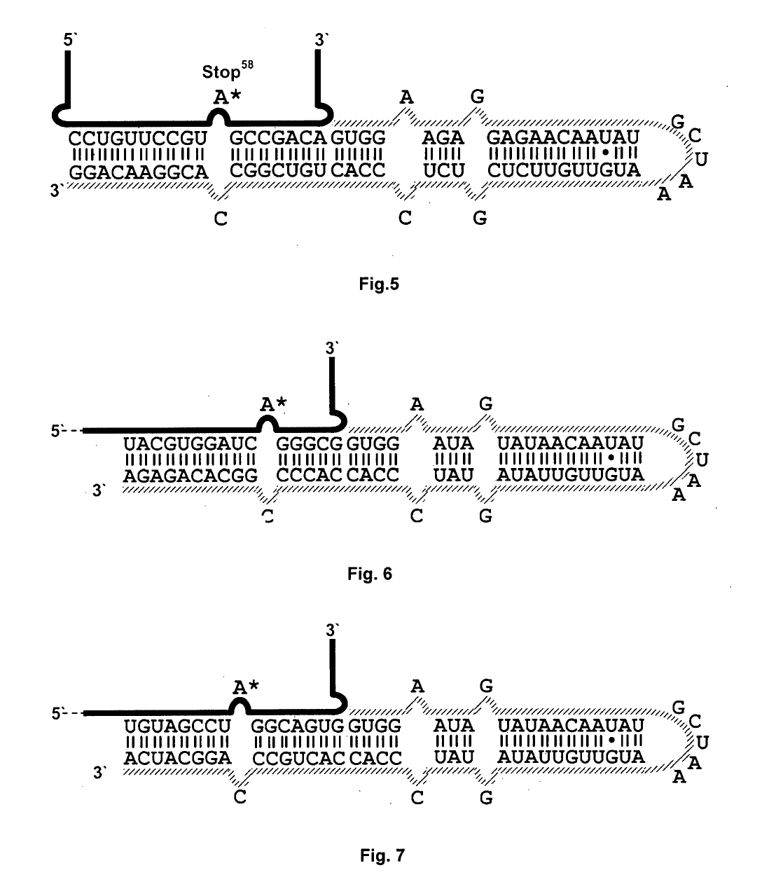

W58X is a mutation in the “TGG” codon and leads to the “TAG” stop codon. The guide RNAs constructed for this exemplary embodiment are illustrated in FIG. 4 and FIG. 5.
a) Standard HEK293T cells as described in the foregoing.
b) HEK293T cells with inducible ADAR2 protein expression (genomically integrated ADAR2). This integrated cell line, in which ADAR2 is induced, has approx. 20-times lower expression of the protein than the cells in the preceding examples, which after transfection each express ADAR2 with 300 ng plasmid. In this example, therefore, the intracellular concentrations of the ADAR2 proteins correspond more closely to those that may be found in a natural cell.
a) The cells were cotransfected with the following plasmids:
   300 ng plasmid that codes for W58X. The plasmid is a pcDNA3.1 vector that includes a CMV promotor for gene expression of W58X;
   300 ng plasmid that codes for hADAR2. The plasmid is a pcDNA3.1 vector that includes a CMV promotor for gene expression of hADAR2;
   1600 ng plasmid that codes for the guide RNA. The plasmid is a 2.1-U6 hygro vector that includes a U6 promotor for transcription of the guide RNA.
b) The cells were cotransfected with the following plasmid and hADAR2 expression was induced with doxycycline:
1300 ng plasmid that codes for the guide RNA.

Both examination by microscope and RNA isolation were used to evaluate whether the editing of the W58X GFP mRNA took place.

Successful editing was evidenced by microscope using fluorescent GFP signal within the cells. The editing result is considered positive if the W58X GFP is edited and therefore corrected. Therefore fluorescent cells seen by microscope denote successful editing.

The cells transfected with the aforesaid plasmids were analyzed in the fluorescence microscope in numerous independent experiments and fluorescent cells were observed, suggesting successful editing. In addition, a positive control, in which the plasmid for the correct wild type GFP was transfected instead of the plasmid for W58X GFP, was analyzed. As expected, fluorescent cells were observed by microscope for the positive control.

The same experiments were conducted as a negative control, wherein either the plasmid that codes for the guide RNA or the plasmid that codes for the ADAR2 enzyme was omitted. There was no GFP in the fluorescence microscope, i.e., no positive editing was detectable. Nor was any editing detected in the sequencing of these RNA isolation specimens, which suggests the specificity of this method.

In the analysis of the isolated RNA, first there was reverse transcription to cDNA, which was multiplied then by PCR (polymerase chain reaction) so that sequencing could then be performed. The ratio of adenine to guanine in the sequencing traces at this target position provided information about how strong the editing was. In the experiment, compared to negative controls, the ratio of adenine to guanine in the cells transfected with the aforesaid plasmids was considerably higher, which suggests successful editing. When using the plasmid that codes for the guide RNA with the SEQ ID no. 1, conduct of part a) of the experiment demonstrated an editing yield of 58%, and with the guide RNA with the SEQ ID no. 3 demonstrated an editing yield of 38%. In the conduct of part b) of the experiment, the guide RNA with SEQ ID no. 1 demonstrated an editing yield of 42% and the guide RNA with SEQ ID no. 3 demonstrated an editing yield of 58%.

### Targeted Editing of a Disease-Relevant Mutation in the PINK1 Gene

The PINK1 gene is linked to Parkinson's disease. In this exemplary embodiment, the so-called R407Q mutation was edited in the “CAG” codon.

FIG. 6 illustrates the guide RNA constructed for this exemplary embodiment.

The cells were cotransfected with the following plasmids:
300 ng plasmid that codes for PINK1 R407Q. The plasmid is a pcDNA3.1 vector having a CMV promotor for gene expression;
300 ng plasmid that codes for hADAR2;
1600 ng plasmid that codes for the guide RNA.

The editing was analyzed by means of RNA isolation. Editing in the cells transfected with all of the aforesaid plasmids reached 35-40% at the target position. In the other exemplary embodiment, the W437X mutation was edited in the “TAG” codon. In this way a disease-relevant phenotype (loss of mitophagy) could be repaired.

FIG. 7 illustrates the guide RNA constructed for this exemplary embodiment.

The cells were cotransfected with the following plasmids:
300 ng plasmid that codes for PINK1 W437X. The plasmid is a pcDNA3.1 vector having a CMV promotor for gene expression;
300 ng plasmid that codes for hADAR2;
1600 ng plasmid that codes for the guide RNA.

The editing was analyzed by means of RNA isolation. Editing in the cells transfected with all of the aforesaid plasmids reached 30% at the target position. In addition, a PINK1 functionality assay was performed in HeLa cells and the loss-of-function phenotype could only be restored (microscopic analysis) if hADAR2 and the guide RNA were transfected. Restoration of mitophagy in HeLa cells could be demonstrated using the microscope.

### Targeted Editing of a Mutation in the Luciferase Gene

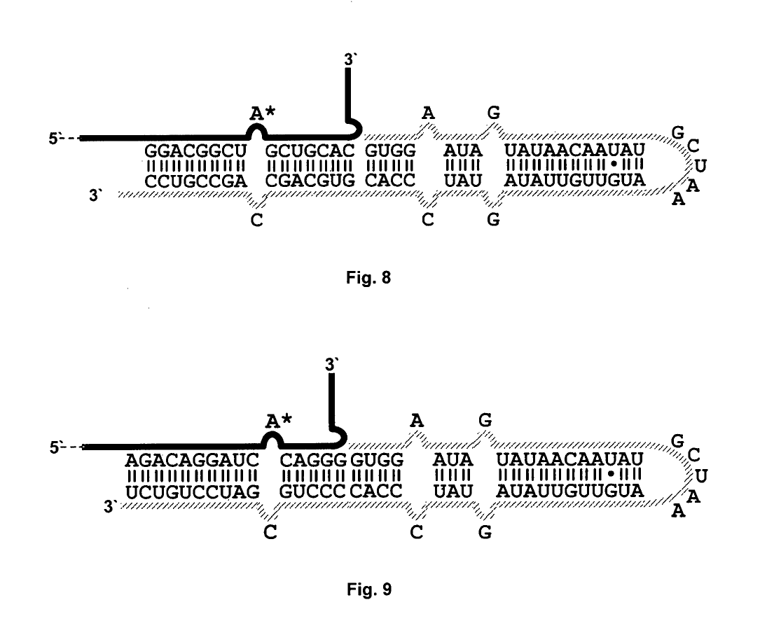

The so-called W417X mutation was edited in the “TAG” codon in this exemplary embodiment. FIG. 8 illustrates the guide RNA constructed for this exemplary embodiment.

The cells were transfected with the following plasmids:
300 ng plasmid that codes for W417X luciferase. The plasmid is a pcDNA3.1 vector having a CMV promotor for gene expression;
300 ng plasmid that codes for hADAR2;
1600 ng plasmid that codes for the guide RNA.

The editing of the W417X luciferase mRNA was analyzed by means of RNA isolation. Editing in the cells transfected with all of the aforesaid plasmids reached 48% at the target position. In the negative control, the plasmid having the specific guide RNA was omitted during the transfection. There was no editing in the negative control, which suggests the exclusivity of the guide RNA in the editing.

### Targeted Editing of a Disease-Relevant Mutation in the FUS Gene

This gene is linked to ALS (amyotrophic lateral sclerosis), a fatal neuro-degenerative disorder. The mutation of this gene is called R521H and includes a “CAC” codon, while the functional FUS has a “CGC” codon.

FIG. 9 illustrates the guide RNA constructed for this exemplary embodiment.

This gene was edited in the PCR reaction vessel. To this end, 350 nM purified ADAR2 protein, 125 nM specific 16 nt long guide RNA, and 25 nM of the mutated R521H FUS mRNA was used. The sequencer result demonstrated that 51% of the adenosines were successfully edited to inosine.

### Targeted Editing of Endogenous Transcripts by Transfecting the Guide RNA

Six different genes (β-actin, GAPDH, GPI, GUSB, VCP, RAB7A) that are expressed endogenously at different strengths were edited. These were not disease-relevant mutations that have been addressed, but instead depict the attainability of naturally expressed mRNAs. The following cells were used for transfection:

a) standard HEK293T cells (as above) and
b) HEK cells having inducible ADAR2 protein expression (genomically integrated ADAR2). This integrated cell line, in which ADAR2 is induced, has approx. 20-times lower expression of the protein than the cells in the foregoing examples that express ADAR2 after transfection at 300 ng plasmid each. In this example, therefore, the intracellular concentrations of the ADAR2 proteins thus correspond more to those found in a natural cell.

The guide RNA structures of the flexible part H+I for the specific target genes are provided in Table 2.

TABLE 2
List of the guide RNAs that were used for
editing endogenous target genes.

| Name of the guide RNA | Sequence of H + I 5′→3′ |
|---|---|
| TAG#1 β-Actin | ACGCAACCAAGUCAUA |
| TAG#3 β-Actin | GCAAUGCCAUCACCUC |
| TAG#1 GAPDH | AGGGGUCCACAUGGCA |
| TAG#2 GAPDH | GGCUCCCCAGGCCCCU |
| TAG#1 GPI | UGCCGUCCACCAGGAU |
| TAG#1 GusB | CAGAUUCCAGGUGGGA |
| TAG#2 GusB | UCCCUGCCAGAAUAGA |
| TAG#1 VCP | CUCCGCCCACCAAAUG |
| TAG#2 VCP | CCCAAACCACAACAGA |
| TAG#3 VCP | ACCCACCCACCCAGGU |
| TAG#1 RAB7A | CUGCCGCCAGCUGGAU |
| TAG#2 RAB7A | AGGGAACCAGACAGUU |

a) The cells were cotransfected with the following plasmids:
300 ng plasmid that codes for hADAR2;
1300 ng plasmid that codes for the guide RNA.

The editing was analyzed by means of RNA isolation. The ratio of adenine to guanine in the cells transfected with these plasmids was considerably higher than in the negative controls, suggesting successful editing.

b) The cells were cotransfected with the following plasmid and hADAR2 expression was induced with doxycycline:
1300 ng plasmid that codes for the guide RNA.

The editing was analyzed by means of RNA isolation. The ratio of adenine to guanine in the cells transfected with these plasmids was considerably higher than in the negative controls, suggesting successful editing.

| Gene | Part a) of experiment | Part b) of experiment |
|---|---|---|
| β-Actin | | |
| TAG#1 | 26% | 16% |
| TAG#3 | 24% | 14% |
| GAPDH | | |
| TAG#1 | 19% | 10% |
| TAG#2 | 20% | 11% |
| GPI | | |
| TAG#1 | 16% | 12% |
| GUSB | | |
| TAG#1 | 10% | 10% |
| TAG#2 | 23% | 18% |
| VCP | | |
| TAG#1 | 23% | 23% |
| TAG#2 | 16% | 15% |
| TAG#3 | 12% | 13% |
| RAB7A | | |
| TAG#1 | 31% | 38% |
| TAG#2 | 28% | 35% |

### REFERENCES

1) T. Stafforst, M. F. Schneider; An RNA deaminase conjugate selectively repairs point mutations. Angew. Chem. 2012, 124, 11329-32.
2) Marius F. Schneider, Jacqueline Wettengel, Patrick C. Hoffmann and Thorsten Stafforst; Optimal guide RNAs for re-directing deaminase activity of hADAR1 and hADAR2 in trans. Nucleic Acids Research, 2014, Vol. 42, No. 10 e87
3) Montiel-Gonzales M. F., Vallecillo-Viejo, I., Yudowski, G. A. and Rosenthal, J. J. (2013) Correction of mutations within the cystic fibrosis transmembrane conductance regulator by site-directed RNA editing. Proceedings of the National Academy of Sciences of the United States of America, 110, 18285-18290
4) Vogel, P. and Stafforst, T. (2014) Site-directed RNA editing with antagomir deaminases—a tool to study protein and RNA function. ChemMedChem, 9, 2021-2025.
5) Nishikura, K. (2010) Functions and regulation of RNA editing by ADAR deaminases. Annual review of biochemistry, 79, 321.
6) Vogel, P., Schneider, M. F., Wettengel, J. and Stafforst, T. (2014) Improving site-directed RNA editing in vitro and in cell culture by chemical modification of the guideRNA. Angewandte Chemie, 53, 6267-6271.
7) Bass, B. L. (2002) RNA editing by adenosine deaminases that act on RNA. Annual review of biochemistry, 71, 817.
8) Stefl, R., Oberstrass, F. C., Hood, J. L., Jourdan, M., Zimmermann, M., Skrisovska, L., Maris, C., Peng, L., Hofr, C. and Emeson, R. B. (2010) The solution structure of the ADAR2 dsRBM-RNA complex reveals a sequence-specific readout of the minor groove. Cell, 143, 225-237.
9) Hanswillemenke, A., Kuzdere, T., Vogel, P., Jékely, G. and Stafforst, T. (2015) Site-Directed RNA Editing in Vivo can be Triggered by the Light-Driven Assembly of an Artificial Riboprotein. Journal of the American Chemical Society, 137, 15875-15881.

1. A guide RNA for targeted RNA editing having at least the following nucleotide segments coupled to one another, listed from the 5′ end:
Segment A: Nucleotide sequence having a length of three to five bases, wherein a guanosine is always in position 1 and a uridine is always in position 2;
Segment B: Nucleotide sequence having a length of three to five bases, wherein an adenosine is always in position 1;
Segment C: Nucleotide sequence having a length of eight to ten bases, wherein a guanosine is always in position 1;
Segment D: Nucleotide sequence UAUGCUAAAUG or UAUGCUCAAUG;
Segment E: Nucleotide sequence having a length of eight to ten bases, wherein a guanosine is always in the last position;
Segment F: Nucleotide sequence having a length of three to five bases, wherein a cytosine is always in the last position;
Segment G: Nucleotide sequence having a length of three to five bases, wherein an adenosine is always in the next-to-the-last position and a cytosine is in the last position;
Segment H: Nucleotide sequence having a length of five to nine bases, wherein a cytosine or a uridine is always in the last position;
Segment I: Nucleotide sequence having a length of eight to twenty bases,
wherein individual nucleotides of the segments A and G, B and F, or C and E pair to form a double helix and the nucleotides of segment D form a hairpin structure.

2. The guide RNA according to claim 1, wherein segment A has the length of four nucleotides, and in particular has the GUGG nucleotide sequence.

3. The guide RNA according to claim 1, wherein segment B has the length of four nucleotides, and in particular has the AAUA nucleotide sequence.

4. The guide RNA according to claim 1, wherein segment C has the length of nine nucleotides, and in particular has the GUAUAACAA nucleotide sequence.

5. The guide RNA according to claim 1, wherein segment E has the length of nine nucleotides, and in particular has the UUGUUAUAG nucleotide sequence.

6. The guide RNA according to claim 1, wherein segment F has the length of four nucleotides, and in particular has the UAUC nucleotide sequence.

7. The guide RNA according to claim 1, wherein segment G has the length of four nucleotides, and in particular has the CCAC nucleotide sequence.

8. The guide RNA according to claim 1, wherein segments H and I are constructed such that, they pair with the mRNA to be edited and place the base to be edited in an A:C mismatch pair.

9. The guide RNA according to claim 1, wherein it contains one of the sequences SEQ ID NO: 1 through SEQ ID NO: 7.

10. The guide RNA according to claim 1, wherein a hairpin structure, especially a BoxB motif, is appended on the 3′ end.

11. A method for directed RNA editing in which the guide RNA according to claim 1 is transfected into the cells in which the RNA editing is to be carried out.

12. The method according to claim 11, wherein the transfection occurs by means of a plasmid that codes for the guide RNA and at least one U6 promotor for the transcription of the guide RNA.

13. A method for targeted alteration of genetic information on the RNA level comprising use of the guide RNA according to claim 1.

14. The method according to claim 13 for the repair of individual point mutations.


SEQUENCE LISTING (7 sequences)

SEQ ID NO: 1 - RNA, 44 nt, Artificial Sequence
  Guide RNA
guggauaguauaacaauaugcumaauguuguuauaguaucccac

SEQ ID NO: 2 - RNA, 45 nt, Artificial Sequence
  Guide RNA
guggasssgsasascaauaugcumaauguugsususgssscccac

SEQ ID NO: 3 - RNA, 45 nt, Artificial Sequence
  Guide RNA
guggaasagsasaacaauaugcumaauguuguususgusucccac

SEQ ID NO: 4 - RNA, 44 nt, Artificial Sequence
  Guide RNA
guggasasuauaacaauaugcumaauguuguuauagyaycccac

SEQ ID NO: 5 - RNA, 45 nt, Artificial Sequence
  Guide RNA
guggasssgssssssssuaugcumaaugssssssssgssscccac

SEQ ID NO: 6 - RNA, 45 nt, Artificial Sequence
  Guide RNA
guggaassgsasascaauaugcumaauguugsususgssucccac

SEQ ID NO: 7 - RNA, 46 nt, Artificial Sequence
  Guide RNA
guggsauaguauaacaauaugcumaauguuguuauaguauscccac